(19) United States
(12) Patent Application Publication
(10) Pub. No.: US 2019/0053760 A1
(43) Pub. Date: Feb. 21, 2019

(54) MAGNETIC RESONANCE IMAGING CANCER PROBE AND METHODS OF USE

(71) Applicant: Rex Ervin Gerald, II, Willow Springs, IL (US)

(72) Inventor: Rex Ervin Gerald, II, Willow Springs, IL (US)

(21) Appl. No.: 15/922,880

(22) Filed: Mar. 15, 2018

Related U.S. Application Data
(60) Provisional application No. 62/471,712, filed on Mar. 15, 2017.

(51) Int. Cl. A61B 5/00; A61B 5/055; H01Q 1/44; H01Q 1/36; A61H 39/08; A61L 31/08; A61L 31/02; G01R 33/28; G01R 33/56; G01R 33/485

(52) U.S. Cl. CPC A61B 5/6848; A61B 5/055; A61B 5/444; A61B 5/4839; H01Q 1/44; G01R 33/485; A61H 39/086; A61L 31/088; A61L 31/022; G01R 33/286; G01R 33/5601; H01Q 1/36

(57) ABSTRACT

A magnetic resonance spectroscopy and imaging device and method of use for accurately delineating the lateral extent and depth of a skin tumor. The device includes a radiofrequency tuned circuit with an antenna in the shape of an acupuncture needle that is positionally-controlled using a mechanical actuator to provide high-contrast spatial images of skin tumors with sub-millimeter lateral and depth resolution. The device is used by dermatologists to provide accurate spatial demarcations of skin tumors, which facilitate the complete excision of a tumor in the first pass of the Mohs micrographic surgical procedure. Different sizes and shapes of the needle antenna provide advantages for sensitivity, image resolution, and capabilities to impact treatments. A hollow needle antenna provides for a capability of the inventive device to affect treatment of a skin tumor by injecting a chemical agent while monitoring chemical and spatial aspects of the tumor, and by aspirating cancer tissue to remove the tumor and related chemical treatment agents.

### CROSS-REFERENCE TO RELATED APPLICATIONS

This application claims priority from U.S. Provisional Patent Application Ser. No. 62/471,712, filed on Mar. 15, 2017.

### GOVERNMENT INTEREST

The United State Government has certain rights in the invention under Contract No. W-31-109-ENG-38 between the U.S. Department of Energy (DOE) and UChicago Argonne, LLC operating Argonne National Laboratory.

### FIELD OF THE INVENTION

The present invention is generally related to the use of magnetic resonance spectroscopy (MRS) and magnetic resonance imaging (MRI) probes used for chemically and spatially characterizing skin tumors. In particular, dermatologists can benefit from accurate chemical identifications and spatial demarcations of the lateral and depth dimensions of basal cell carcinomas before excision of tumors, resulting in highly-effective single-pass Mohs micrographic surgeries. The present invention further provides capabilities to inject treatment agents into tumors and extract tumor cells before, during, and after recording MRS/MRI images.

### BACKGROUND OF THE INVENTION

The inventive device broadly relates to the field of cancer in humans and the tools that personnel in the medical profession need to identify and spatially delineate cancer tumors that are slated for surgical removal. The inventive device specifically relates to the field of skin cancers and the Mohs microsurgery procedure, the most effective method for treatment of basal cell carcinomas. The inventive device more specifically relates to the field of nuclear magnetic resonance (NMR) spectroscopy and magnetic resonance imaging (MRI) probes that are used to identify and spatially delineate cancer tumors to improve the Mohs microsurgery procedure. The inventive device more specifically relates to the field of magnetic resonance spectroscopy (MRS) and MRI surface coil probes that are used to identify and spatially delineate skin tumors as small as one cubic millimeter in size to accurately direct the Mohs microsurgery procedure to an effective treatment outcome. The inventive device more specifically relates to the use of an MRS/MRI probe comprised of a sensor element that has three functions: (1) Radiofrequency sensing of MRS/MRI signals; (2) Near non-invasive piercing and positioning in soft matter; (3) Injection of cancer treatment agents into tumors and removal of tumor cells. Most specifically, one embodiment of the inventive device, the Acupuncture-MRI Probe, comprises an acupuncture needle as a portion of the inductor element of a radiofrequency tuned circuit. The acupuncture needle serves three functions: (1) An antenna for excitation and detection of nuclear spins in human tissue; (2) A tapered rod for piercing human tissue to position said antenna in proximity to cancer cells; (3) A hollow needle for injecting cancer treatment agents and aspirating tumor cells and associated deleterious disease materials.

The most common cancer, with 5.4 million cases in the US in 2012, is nonmelanoma skin cancer (NMSC), comprised of basal cell carcinoma (BCC) and squamous cell carcinoma (SCC). The estimated cost to treat NMSC is $8.1 billion annually. The most effective therapy for NMSC is Mohs micrographic surgery (Mohs). The average cost of treatment per patient for NMSC increased from $1000 in 2006 to $1600 in 2011, largely due to the 700% increase in the rate of Mohs; Mohs procedures typically have Medicare payments 120% to 370% more than surgical excision, even when including pathology fees. The Mohs procedure entails costly and time-consuming repeat stages of excisions and microscopic examinations (1^{st} pass, 2^{nd} pass, etc.), so that patients, often in their 80s and 90s, must sometimes wait hours before several passes yield a clear field. Yet recurrences are still in the range of 1-5% of Mohs-treated cases. New methods are needed to improve NMSC treatment outcomes by reducing treatment time, expense, treatment failure, pain, and suffering.

The ability of instruments to probe depth is crucial for the determination of residual tumor, especially in the perinasal and periocular areas, where loss of a vital organ may be the penalty for failure to remove all tumor cells. A device that is capable of spatially mapping subsurface tumors in the skin has the potential to become the gold standard for accurate skin cancer demarcation. Accurate spatial mapping of skin tumors is a key requirement for subsequent complete tumor excision.

Non-invasive in vivo technologies for accurate skin-cancer demarcation are limited in either measurement depth or sensitivity. Since skin cancers originate in the epidermis, they are amenable to sensitive optical methods that provide molecular signatures. However, optical detection is limited by its depth of screening due to the light scattering and absorption of light by tissues. Relevant optical methods include confocal microscopy/spectroscopy of Raman scattering, near infra-red (NIR), including NIR fluorescence, and visible light. The depth limit is typically 1 mm for Raman scattering, 0.3-0.5 mm for NIR, and less than 0.7 mm for visible confocal microscopy. Surface-enhanced Raman spectroscopy (SERS) provides greater sensitivity than regular Raman but the nanoparticles used for signal enhancement typically do not penetrate far into the skin, and thus penetration depth of SERS is limited to only a few microns. Therefore, the primary limitation of optical microscopy/spectroscopy methods is the limited penetration depth of 1 mm or less from the surface of the skin. There is a need for an imaging device that can easily probe depths of 0.5-8 mm below the surface of the skin, the range necessary to detect both early and more advanced skin tumors.

Magnetic resonance spectroscopy (MRS) has the inherent capability to detect molecular-level signatures in cancer tissues (e.g., choline, lactate, alanine, lipids, N-acetyl aspartate). However, the drawback of MRS is its overall low level of sensitivity and concomitant low imaging resolution, especially for small cancer detection. In clinical settings, MRS traditionally uses the ¹H- and ³¹P nuclei. To obtain a sufficiently high signal-to-noise (SNR) ratio, it is necessary for voxels to be large (1-8 cm³), which limits the ability to define small tumor volumes (<1 mm³) precisely. Enhancement of MRS signals has been achieved with hyperpolarization techniques such as dynamic nuclear polarization (DNP). However, DNP requires excited electronic states which can lead to tissue heating and the destruction of sensitive bioorganic molecules. The half-lives of DNP induced hyperpolarizations are relatively short, and there are currently technical challenges to incorporate a polarizer in a clinical setting. Chemical exchange saturation transfer has also been used in MRI to enhance contrast and detect individual metabolites, but disadvantages include reliance on protons that exchange on the slow-to-intermediate timescale, interferences with H₂O or other proton-exchanging groups, and the nuclear Overhauser effect. What is needed in the field of surgical removal of cancer tissue is an MRS probe that is inherently more sensitive than traditional MRS surface coil probes because it can be placed in much closer proximity to the location of the tumor. It would be further advantageous if a new MRS probe design would provide for a means to easily combined the probe technology with emerging hyperpolarization techniques. More specifically, it would be highly advantageous if a new MRS probe design would incorporate a means for delivering hyperpolarized fluids directly to the location of the tumor, providing opportunities for opening additional critical avenues for recording high-sensitivity and high-contrast MRI images of treatment processes not obtainable with current hyperpolarization-based MRI technology alone.

Single-pass Mohs micrographic surgery outcomes and costs could be improved with a sensitive high-resolution MRS/MRI device that accurately delineates NMSC molecular signature margins. The design of a new MRS/MRI imaging device would include the following advantages: (1) minimally invasive to human tissues; (2) capable of providing accurate delineation of skin cancer margins with less than 0.5 mm error on lateral (in-plane) and depth margins; (3) enhanced sensitivity for MRS/MRI signals due to proximity to the suspect tumor; and (4) 100-fold sensitivity enhancement over traditional MRS.

### BRIEF SUMMARY OF THE INVENTION

The present invention relates to a novel magnetic resonance imaging and spectroscopy (MRI/MRS) probe. The Acupuncture-MRI/MRS Probe is one embodiment of the present invention and consists of: (a) an acupuncture-needle antenna that functions as an inductor in a radiofrequency resonant circuit; (b) multiple non-magnetic capacitor tuning/matching elements that function to tune and match the resonant circuit; and (c) a mechanical actuator that functions as an accurate positioning device for the acupuncture-needle antenna. In a series of discreet steps controlled by the mechanical actuator, the acupuncture-needle antenna penetrates a skin tumor slated for excision and provides high-contrast MRI/MRS images that accurately delineate the tumor with sub-millimeter lateral and depth resolution. The novel Acupuncture-MRUMRS (A-MRI/MRS) Probe invention enables 100-fold higher magnetic resonance sensitivity, due to the proximity of the needle antenna to the tumor, compared to the most sensitive commercially available MRI/MRS surface-coil probes. The enhanced sensitivity provided by the inventive device is useful for recording high-resolution magnetic resonance images, which would enable dermatologists to accurately and completely excise tumors in the first pass of the Mohs microsurgery procedure. The inventive device improves skin cancer treatment outcomes by reducing surgery time, expense, failure rate, pain, and suffering.

### BRIEF DESCRIPTION OF THE SEVERAL VIEWS OF THE DRAWING

FIG. 1 shows a side view of one embodiment of the Acupuncture-MRI/MRS Probe.

FIG. 2 shows a graph containing a plot of Chemical Shift (ppm) versus Nutation Frequency (Hz) versus NMR Signal Intensity (a.u.).

FIG. 3 shows a graph containing a plot of Chemical Shift (ppm) versus Position (mm) versus NMR Signal Intensity (a.u.).

FIG. 4 shows a graph containing a plot of A-MRI Needle Tip Position from Skin Surface (mm) versus Integral of Proton NMR Signal (a.u.).

FIG. 5 shows a graph containing a plot of A-MRI Needle Tip Position from Skin Surface (mm) versus Derivative of Integral of Proton NMR Signal (a.u.).

### DETAILED DESCRIPTION OF THE INVENTION

One embodiment of the present invention relates to an Acupuncture-MRI Probe having a slender tapered metallic rod that simultaneously serves as the antenna of a radiofrequency resonant circuit and a needle for piercing skin tissues. The proximity of the MRI detector needle antenna to subcutaneous skin tumors provides up to a 100-fold sensitivity enhancement for magnetic resonance signals compared to MRI surface coils known in the art. The enhanced magnetic resonance signals are suitable for spatially mapping the margins of basal cell carcinomas with sub-millimeter resolution. The acupuncture needle antenna is electrically connected to capacitors to form a radiofrequency resonant circuit and a mechanical actuator to provide accurate positioning in skin tissues. The Acupuncture-MRI Probe is connected to a radiofrequency electrical console that generates a radiofrequency electrical current in the needle antenna. The radiofrequency electrical current generates an oscillating magnetic field that is used to interrogate nuclear spins, contained in the atoms that make up skin tumors, when they are placed in a large static magnetic field. Magnetic resonance signals from skin tumors are detected by the needle antenna following short radiofrequency interrogation pulses. The signals are transferred to the radiofrequency electrical console and processed into images by a computer. High-resolution images of skin tumors, derived from magnetic resonance signals recorded by the Acupuncture-MRI Probe, are used to accurately demarcate a perimeter of tissue for excision. The excised tissue undergoes histopathological examination according to the Mohs microsurgery procedure. The Acupuncture-MRI Probe guides the Mohs microsurgery procedure so that only a single pass excision is required to achieve complete removal of skin tumors and a corresponding 100% effective treatment rate.

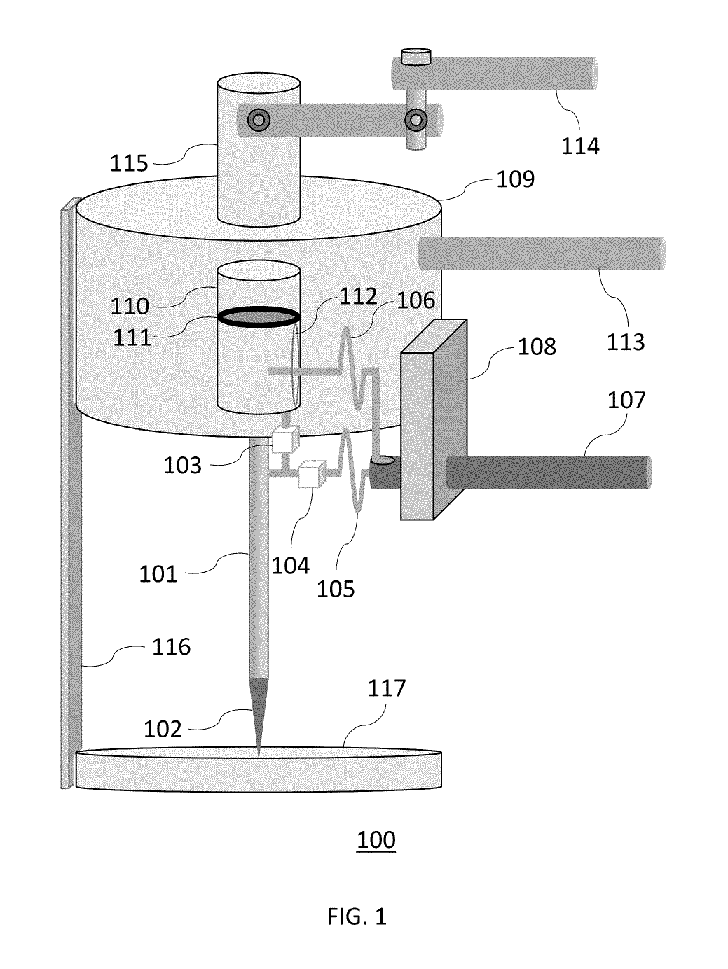

FIG. 1 shows one embodiment of the Acupuncture-MRI Probe 100 having a radiofrequency needle antenna 101 fitted with a tapered point 102. The tapered point 102 is useful for piercing skin tumors to position needle antenna 101 proximate to cancer cells. A suitable diameter of needle antenna 101 is 150 μm for skin tissues that are calloused and stiff. A suitable diameter of the needle antenna 101 is 100 μm for normal skin tissues. A suitable diameter of the needle antenna is 50 μm for soft skin tissues.

A non-magnetic ceramic capacitor 103 is electrically connected to needle antenna 101 and employed to tune needle antenna 101 to resonant radiofrequencies suitable for interrogation of nuclear spins in skin tumors. A capacitance for non-magnetic ceramic capacitor 103 ranges from 0.001 to 100 pF. A capacitance value of up to 100 pF is suitable for nuclei that resonate at low frequencies (e.g., ¹⁵N, ⁵⁷Fe, ¹⁰³Rh) A capacitance value of up to 20 pF is suitable for nuclei that resonate at medium frequencies (e.g., ⁷Li, ³¹P, ¹¹⁹Sn). A capacitance value of up to 5 pF is suitable for nuclei that resonate at high frequencies (e.g., ³H, ¹H, ¹⁹F).

A non-magnetic ceramic capacitor 104 is electrically connected to needle antenna 101 and employed to match the tuned circuit to the 50Ω output impedance of a radiofrequency amplifier. A capacitance value for non-magnetic ceramic capacitor 104 of up to 20 pF is suitable for nuclei that resonate at low frequencies (e.g., ¹⁵N, ⁵⁷Fe, ¹⁰³Rh) A capacitance value of up to 2 pF is suitable for nuclei that resonate at medium frequencies (e.g., ⁷Li, ³¹P, ¹¹⁹Sn). A capacitance value of up to 1 pF is suitable for nuclei that resonate at high frequencies (e.g., ³H, ¹H, ¹⁹F). Match non-magnetic ceramic capacitor 104 is connected to needle antenna 101, non-magnetic ceramic tune capacitor 103, and to flexible electrical conductor 105.

Flexible electrical conductor 105 is electrically connected to radiofrequency signal cable 107. Radiofrequency signal cable 107 is rigidly fixed to support structure 108. The electrically-grounded shielding conductor of radiofrequency signal cable 107 is electrically connected to flexible electrical conductor 106. Flexible electrical conductor 106 is electrically connected to tune non-magnetic ceramic capacitor 103 and needle antenna 101 through elongated through hole slot 112.

Mechanical actuator 109 receives electrical control signals through electrical cable 113, which cause electromechanical piston 111 to accurately position vertically in hollow tube guide 110. Needle antenna 101 is rigidly connected to electromechanical piston 111. The position of electromechanical piston 111 and needle antenna 101 are under computer control.

The stroke length of electromechanical piston 111 and attached needle antenna 101 ranges from 0.01 to 20 mm. A suitable stroke length is 15 mm for many general imaging applications. A stroke length is 10 mm is suitable for most skin tumors. The stroke length of 3 mm is approximately commensurate with the average thickness of the dermis layer, 2-3 mm, and is suitable for most small skin tumors.

Position control arm 114 provides for precise horizontal and vertical placement control of needle antenna 101 on the surface of subject's skin. The position control arm 114 is rigidly connected to mounting post 115. Mounting post 115 is rigidly connected to mechanical actuator 109. Support structure 116 is rigidly connected to mechanical actuator 109 and ring stop 117. Ring stop 117 is made to contact surface of subject's skin, using position control arm 114, to define a perimeter around skin tumor.

In operation, Acupuncture-MRI Probe 100 is positioned above the surface of subject's skin tumor using position control arm 114. Fine adjustments of position control arm 114 are employed to position ring stop 117 concentric with skin tumor. Mechanical actuator 109 receives electrical control signals through electrical cable 113, which cause electromechanical piston 111 to position vertically in hollow tube guide 110 accurately. Needle antenna 101 is rigidly connected to electromechanical piston 111, and at the start of the process for imaging the skin tumor, is positioned in direct contact with the skin's surface above the tumor.

In one method of operation of the Acupuncture-MRI Probe 100, the procedure for generating an image of the skin tumor and surrounding tissue includes that needle antenna 101 is made to pierce and take a stationary position in the skin tumor such that tapered point 102 extends approximately 0.5 mm below the dermis layer. A radiofrequency pulse is applied to the resonant circuit that comprises needle antenna 101, tune non-magnetic ceramic capacitor 103, and match non-magnetic ceramic capacitor 104. The radiofrequency pulse causes a momentary excitation of the resonant nuclei in the skin tumor and surrounding tissue. Subsequently, needle antenna 101 detects radiofrequency signals from nuclei from skin tumor and surrounding tissue. A radiofrequency console connected to the resonant circuit via radiofrequency signal cable 107 records the radiofrequency signals. External static magnetic field gradient coils that are standard components of MRI scanners are pulsed, according to specific imaging protocols known in the art of MRI, during the acquisition of the radiofrequency signals by the Acupuncture-MRI Probe 100. Computer algorithms known in the art of MRI are used to process the radiofrequency signals into an image of the skin tumor and surrounding tissue.

In another method of operation of the Acupuncture-MRI Probe 100, the procedure for generating an image of the skin tumor and surrounding tissue includes that needle antenna 101 is made to pierce and take a stationary position in the skin tumor such that tapered point 102 extends approximately 0.5 mm below the dermis layer. Needle antenna 101 is energized with a sequence of radiofrequency pulses; after each radiofrequency pulse in the sequence, the radiofrequency signals from the nuclei in the skin are detected and stored in a computer. The first radiofrequency pulse in the sequence always has a duration of 0 μs. Subsequent pulses have durations that are whole number increments of a pulse duration termed the dwell time for a one-dimensional image coordinate. The sequence of radiofrequency pulses consists of 2ⁿ (n˜1-16) individual pulses of different lengths. The one-dimensional image coordinate for the Acupuncture-MRI Probe 100 is the radial coordinate of a cylindrical coordinate system with the center axis coincident with the long axis of needle antenna 101. The radiofrequency signals collected and stored following each radiofrequency pulse are individually Fourier transformed from the time to the frequency domain. The resultant series of spectra are ordered into data point arrays, and Fourier transformed to produce a data set that constitutes a one-dimensional in-plane radial image of the skin tumor. The procedure for eliciting, detecting, and performing a double Fourier transform of the collected and stored radiofrequency signals from nuclei in phantom test samples, using home-built coils of various designs and commercially-available surface coils, and generating a one-dimensional image is known in the art as Rotating Frame Imaging (RFI). The Acupuncture-MRI Probe 100 uses the RFI method to generate one-dimensional in-plane radial images (radial profiles) of skin tumors. A typical sequence of radiofrequency pulses consists of 4096 pulses of increasing durations, starting with a pulse duration of 0 μs and having a pulse duration increment of 5 μs. The RFI procedure for producing one-dimensional radial images using toroid cavity detector probes, which generate cylindrically-symmetric radiofrequency magnetic fields, is well established and known in the art; the Acupuncture-MRI Probe 100 invention incorporates the RFI procedure for recording images using toroid cavity detector probes. The Acupuncture-MRI Probe 100 invention is distinguishable from toroid cavity detector probes by its open architecture; the limitation of toroid cavity probes is that they are closed-container probes that are too small to contain or accommodate the human body or human appendages. The Acupuncture-MRI Probe 100 invention exhibits open access to the needle antenna 101 so that the human body and appendages can be accommodated for analyses of skin tumors.

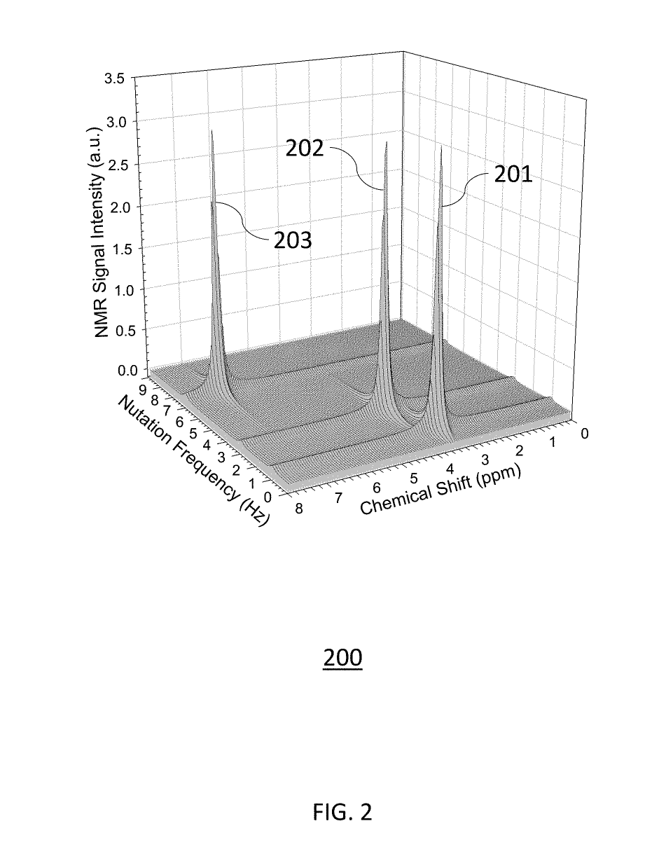

In another example, the RFI method for imaging skin tumors includes a sequence of radiofrequency pulses that consists of 256 pulses of increasing durations, starting with a pulse duration of 0 μs and having a pulse duration increment of 50 ms. FIG. 2 shows a graph 200 containing a plot of Chemical Shift (ppm) versus Nutation Frequency (Hz) versus NMR Signal Intensity (a.u.). The plot constitutes a computer-simulated one-dimensional radial image (radial profile) generated by a representative embodiment of the Acupuncture-MRI Probe 100 invention. The protocol for collecting the radiofrequency signals from nuclei and processing the data with a double Fourier transform was described above. The one-dimensional radial image comprises a map of resonance frequencies, or equivalently chemical shifts, of nuclei in the skin tumor and surrounding tissue, their corresponding nutation frequencies, and the quantities of nuclei that give rise to each one of the resonance frequencies. The nutation frequency for each radiofrequency signal is inversely proportional to the radial position of the nuclei that give rise to the corresponding radiofrequency signal. Specifically, a first group of nuclei gives rise to normal skin tissue radial image feature 201, which has a chemical shift of 3.5 ppm and a nutation frequency of 1 Hz; this group of nuclei is furthest from needle antenna 101. A second group of nuclei gives rise to predominantly normal skin tissue radial image feature 202, which has a chemical shift of 4 ppm and a nutation frequency of 3 Hz; this group of nuclei is closer to needle antenna 101. A third group of nuclei gives rise to skin cancer tissue radial image feature 203, which has a chemical shift of 7 ppm and a nutation frequency of 7 Hz; this group of nuclei is closest to needle antenna 101. The RFI image in FIG. 2 indicates to the dermatologist that skin cancer tissue radial image feature 203, which has a chemical shift of 7 ppm and a nutation frequency of 7 Hz should be excised.

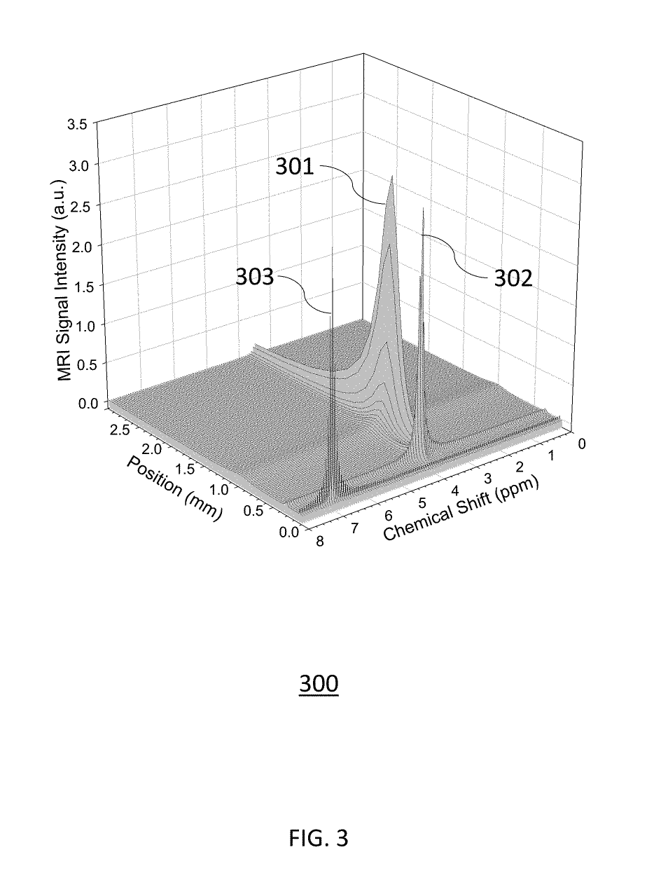

FIG. 3 shows a graph 300 containing a plot of Chemical Shift (ppm) versus Position (mm) versus NMR Signal Intensity (a.u.). The plot constitutes a computer-simulated one-dimensional radial image (radial profile) generated by a representative embodiment of the Acupuncture-MRI Probe 100 invention. The protocols for collecting the radiofrequency signals from nuclei and processing the data with a double Fourier transform were described above. The one-dimensional radial image comprises a map of resonance frequencies, or equivalently chemical shifts, of nuclei in the skin tumor and surrounding tissue, their corresponding radial positions, and the quantities of nuclei that give rise to each one of the resonance frequencies. The RFI image in FIG. 3 shows three distinct groups of skin tissue nuclei. Specifically, a first group of nuclei gives rise to normal skin tissue radial image feature 301, which has a chemical shift of 3.5 ppm and a radial position of 1.00 mm; this group of nuclei is furthest from needle antenna 101. A second group of nuclei gives rise to predominantly normal skin tissue radial image feature 302, which has a chemical shift of 4 ppm and a radial position of 0.333 mm; this group of nuclei is closer to needle antenna 101. A third group of nuclei gives rise to skin cancer tissue radial image feature 303, which has a chemical shift of 7 ppm and a radial position of 0.143 mm; this group of nuclei is closest to needle antenna 101. The RFI image in FIG. 3 indicates to the dermatologist that skin cancer tissue radial image feature 303, which has a chemical shift of 7 ppm and a radial position of 0.143 mm should be excised.

Methods described above for obtaining RFI images using inventive device Acupuncture-MRI Probe 100 provide one-dimensional radial in-plane maps of normal and cancer tissues. One-dimensional radial profiles from the centers of skin tumors are suitable for demarcating most skin tumors because they are typically round structures. An extension of the RFI methods described above provides for measurements of the depth profiles of skin tumors. Thus, in another method of operation of the Acupuncture-MRI Probe 100, the procedure for generating an image of a skin tumor and surrounding tissue, with radial and depth profiles, includes the placement of needle antenna 101 at a stationary position in the skin tumor such that tapered point 102 extends approximately 0.1 mm below the skin's surface. Needle antenna 101 is energized with a complete sequence of radiofrequency pulses. Following each complete sequence of radiofrequency pulses, the radiofrequency signals from the nuclei in the skin are detected, stored in a computer, and processed into a one-dimensional radial image, as described above. Next, the step-wise positioning of needle antenna 101 deeper into the skin tumor, under computer control, proceeds in increments of 100 μm, and the RFI protocol is repeated to generate the next one-dimensional radial image. Alternatively, positioning increments of 10 μm may be used for finer depth resolution of the skin tumor. Conversely, positioning increments of 200 μm may be used for coarser depth resolution of the skin tumor. The collection of RFI images as a function of depth position in the skin tumor is analyzed, using known methods in the art, to produce a depth profile of the skin tumor.

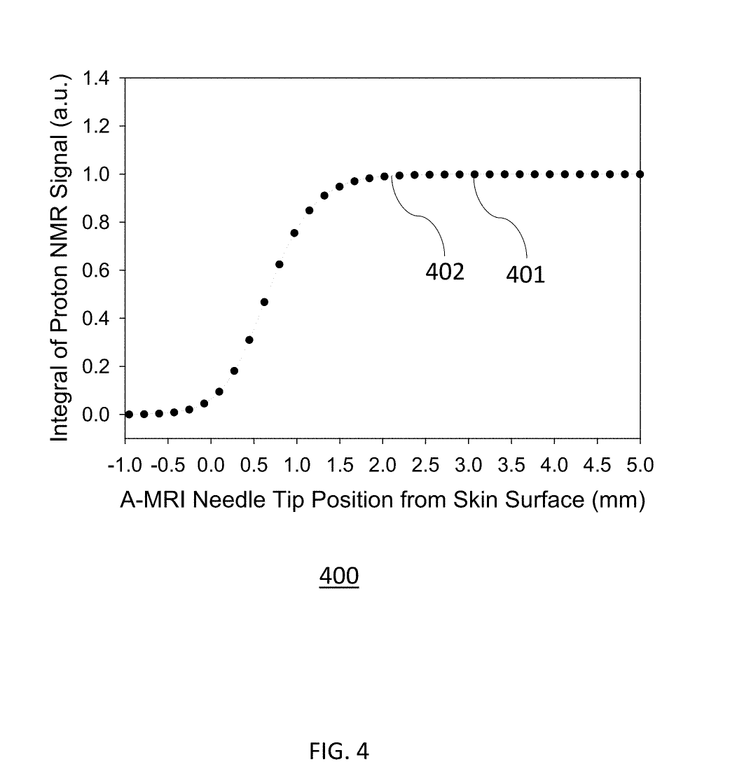

FIG. 4 shows a graph 400 containing a plot of A-MRI Needle Tip Position from Skin Surface (mm) versus Integral of Proton NMR Signal (a.u.). For illustration purposes, the computer-simulated plot provides a depth profile map using the integral of proton NMR signal from skin cancer tissue radial image feature 303 in FIG. 3 from a series of RFI images. Each depth profile data point 401 in the plot in FIG. 4 graph 400 derives from the integral of the NMR signal intensity of skin cancer tissue radial image feature 303, which has a chemical shift of 7 ppm and a radial position of 0.143 mm from the needle antenna 101 and corresponds to a skin tumor penetration depth of needle antenna 101. The solid curve 402 in the plot in FIG. 4 graph 400 represents a best fit to the depth profile data points 401 and shows that the integral of the NMR signal intensity of skin cancer tissue is zero when needle antenna 101 is above the skin tumor, reaches a maximum when needle antenna 101 completely penetrates the skin tumor, and maintains a maximum value when the needle antenna extends to skin depths beyond the skin tumor.

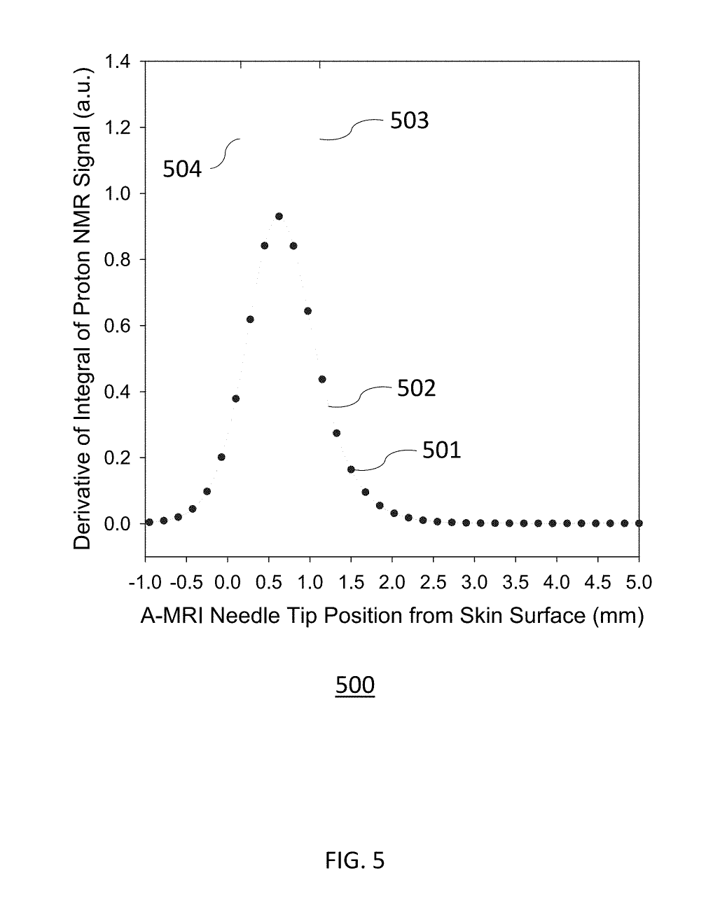

FIG. 5 shows a graph 500 containing a plot of A-MRI Needle Tip Position from Skin Surface (mm) versus Derivative of Integral of Proton NMR Signal (a.u.), which was derived from the data in FIG. 4 graph 400. Each depth profile data point 501 in the plot in FIG. 5 graph 500 derives from the derivative of the integral of the NMR signal intensity of skin cancer tissue radial image feature 303, which has a chemical shift of 7 ppm and a radial position of 0.143 mm from the needle antenna 101 and corresponds to a skin tumor penetration depth of needle antenna 101. The solid curve 502 in the plot in FIG. 5 graph 500 represents a best fit to the depth profile data points 501 and shows that the derivative of the integral of the NMR signal intensity of skin cancer tissue is zero when needle antenna 101 is above the skin tumor, reaches a maximum at approximately 0.6 mm when needle antenna 101 completely penetrates the skin tumor, and reverts to a minimum value when the needle antenna extends to skin depths beyond the skin tumor. Top demarcation skin tumor line 504 at approximately 0.15 mm shows the approximate position of the top of the skin tumor near the surface of the skin. Bottom demarcation skin tumor line 503 at approximately 1.1 mm shows the approximate position of the bottom of the skin tumor below the surface of the skin.

The RFI image in FIG. 3 and the depth profile image in FIG. 5 indicate to the dermatologist that skin cancer tissue radial image feature 303, which has a chemical shift of 7 ppm, a radial position of 0.143 mm, and a depth profile below the surface of the skin that spans from 0.15 to 1.1 mm should be excised.

As discussed above, the Acupuncture-MRI Probe 100 described herein takes advantage of the fundamental magnetic-resonance tenant that proximity of the detector to the sample is the key factor in overall measurement sensitivity. Early-stage detection of skin cancers using the Acupuncture-MRI Probe 100 described herein can include a non-invasive or minimally invasive structure of conducting material as the radiofrequency antenna generating a localized radiofrequency magnetic field with gradient distribution radially surrounding the needle antenna 100. It should be understood that the Acupuncture-MRI Probe 100 depicted in FIG. 1 is just an example probe, and the broader structural and inherent properties of the Acupuncture-MRI Probe 100 and associated parameters contemplated by this invention are discussed herein. A probe system not described herein that includes a micro-engineered needle micro-coil for MRI that can record in vivo ³¹P MRS was disclosed by Syms et al. (see R. Syms, M. Ahmad, I. Young, D. Gilderdale, and D. Collins, “Microengineered needle micro-coils for magnetic resonance spectroscopy,” Journal of Micromechanics and Microengineering, vol. 16, p. 2755, 2006, and F. A. Howe, R. R. Syms, M. M. Ahmad, L. M. Rodrigues, J. R. Griffiths, and I. R. Young, “In vivo 31p magnetic resonance spectroscopy using a needle microcoil,” Magnetic resonance in medicine, vol. 61, pp. 1238-1241, 2009.) However, a major deficiency of the device described by Syms et al. is that the micro-coil needle is brittle and therefore can break off while it is inserted into the subject. Another deficiency of the Syms et al. invention is that the size of the micro-coil needle must be larger than 1.5 mm to prevent the silicon substrate from breaking; the large size of the brittle silicon needle causes substantial pain and discomfort when inserted into the subject. The present invention is patentable over the micro-coil invention by Syms et al. because the size of the Acupuncture-MRI Probe needle antenna 101 described herein is substantially less than the 1.5 mm micro-coil needle size of Syms and others. The Acupuncture-MRI needle antenna 101 has a diameter that is at least ten times smaller compared to the micro-coil needle size of Syms et. al., as described further below, thereby allowing for a non-invasive or minimally invasive use with subjects for diagnostic and treatment purposes. In addition, the Acupuncture-MRI Probe 100 described herein can generate and detect localized radiofrequency magnetic fields along a radial coordinate, which affords the capability to elicit and collect signature signals from a small cluster of cancer cells making early-stage skin cancer detection feasible. Furthermore, the Acupuncture-MRI Probe 100 described herein is a very localized and quiet sensor; it does not require the application of large pulsed magnetic field gradients, which cause loud and disturbing clicking sounds that patients find objectionable. Also, the radiofrequency field is highly localized, so that interference with pacemakers and metal implants is negligible. In certain aspects, the Acupuncture-MRI Probe 100 disclosed herein affords enhanced sensitivity to detect early-stage disease and provides a high-resolution (<10 μm) radial profile and a depth profile (ca. 100 μm resolution) of the diseased skin tissue. While, in aspects, the Acupuncture-MRI Probe 100 may be mildly invasive, it offers the advantages of very high sensitivity, chemical specificity, and micrometer resolution. The capabilities of the Acupuncture-MRI Probe 100 described here can be considered a combination of non-invasive standard MRI/MRS technology with the attributes of invasive biopsy resulting in treatment outcomes with minimal scar tissue. Significantly, the Acupuncture-MRI Probe 100 is patentable over the micro-coil invention by Syms et al. because in one embodiment of the present invention, the needle antenna 101 can comprise a hollow electrically conductive tube that can be effectively used to inject imaging and treatment agents and compounds into the tumor. The injection process can proceed before, during, and after the RFI process. That is, RFI and MRI images of the skin tumor may be collected, viewed, and analyzed before, during, and after the process of injecting imaging and treatment agents and compounds into the skin tumor. Furthermore, a needle antenna 101 in the form of a hollow electrically-conductive tube can have perforated tube walls. The perforated tube walls provide means for egress of imaging and treatment agent and compounds. The Acupuncture-MRI Probe 100 invention is further patentable over MRI coils known in the art because a needle antenna 101 in the form of a hollow electrically-conductive tube can be employed to aspirate cancer cells and deleterious materials in skin tumors. The process of removal of cancer-related materials from a skin tumor may proceed before, during, and after the RFI and MRI imaging process thereby affording real-time monitoring of cancer treatment procedures.

In certain aspects, the Acupuncture-MRI Probe 100 comprises a resonating element that can include a continuous wire 101 or conductive material that can be linear, nonlinear, circular, or a combination thereof. In certain aspects, Acupuncture-MRI Probe 100 can include a wire 101 that extends linearly and then shifts into a coiled form to provide an appropriate level of inductance compactly. In certain aspects, the Acupuncture-MRI Probe needle antenna 101 can exhibit an inductance of about 10⁻⁶ to about 10⁻¹² henry (H), or about 100 nH to about 10 μH.

The Acupuncture-MRI Probe needle antenna 101 can comprise any material(s) as long as the Acupuncture-MRI Probe needle antenna 101 exhibits an inductance suitable for use in the methods described herein, such as the inductance levels discussed above. In certain aspects, the Acupuncture-MRI Probe needle antenna 101 can exhibit a rigidity such that the probe can allow for insertion into a subject's tissue. A non-limiting list of example materials that can be used singly or in combination with the Acupuncture-MRI Probe needle antenna 101 includes nonmagnetic stainless steel, phosphor bronze, or glass material (hollow or solid). In certain aspects, the Acupuncture-MRI Probe needle antenna 101 can also include a nonconductive material coated with a conductive material, such as gold, where the coating can range from about 1 μm to about 50 μm or from about 10-20 μm. The Acupuncture-MRI Probe needle antenna 101 can be single use or re-usable as long as the re-usable needle antenna 101 can be effectively sterilized. In one or more aspects, the Acupuncture-MRI Probe needle antenna 101 may be coated with a commercially available antiseptic material. In various aspects, the Acupuncture-MRI Probe needle antenna 101 can be a hollow structure, similar to a syringe needle, to allow for the deposition of one or more therapeutic agents. In such aspects, the therapeutic agents can include pharmacological compositions alone or pharmacological composition that may be present in glass beads (or another carrier) (e.g., having a diameter of from about 1-50 microns) for deposition in the treatment target tissue where the pharmacological composition may diffuse out of the beads. In alternative aspects, the therapeutic agents can include metal nanoparticles for deposition in the target tissue, where a specific radiofrequency pulse can be used to affect cellular necrosis or apoptosis. In such alternative aspects, the specific radiofrequency pulses required can be determined by one skilled in the art.

In various aspects, the Acupuncture-MRI Probe needle antenna 101 can have a thickness ranging from about 5 μm to about 1000 μm, or from about 10 μm to about 200 μm, or from about 30 μm to about 60 μm, or about 50 μm. It should be understood that the diameter values of the Acupuncture-MRI Probe needle antenna 101 listed above are exemplary, and other diameters may be utilized as long as the probe can function as a resonating element.

In certain aspects, the Acupuncture-MRI Probe needle antenna 101 can have a length from about 1 mm to about 150 mm, or from about 5 mm to about 75 mm. It should be understood that such length values are exemplary and not limiting since in various aspects, the Acupuncture-MRI Probe needle antenna 101 can vary in length depending upon the desired location of the probing target (e.g., a tumor). For example, in one aspect, the probing target may be a group of skin cells suspected as being malignant, in which case the Acupuncture-MRI Probe needle antenna 101 may be at least a few millimeters (e.g., at least about 3 mm) to probe into or around the epidermis where the suspect group of skin cells is located. In such aspects, the Acupuncture-MRI Probe needle antenna 101 can be longer to facilitate handling or connection to one or more non-magnetic ceramic capacitors 103 and 104 or other components discussed herein. In alternative aspects, the probing target may be an internal organ, in which case the Acupuncture-MRI Probe needle antenna 101 can be at least about 50 mm, at least about 100 mm, or at least about 350 mm.

In one or more aspects, the Acupuncture-MRI Probe needle antenna 101 can include a non-metallic tip 102, e.g., a ceramic tip 102, to restrict or turn off the radio frequency (RF) field in the MRI probe at the location of the ceramic or non-metallic tip 102. In one aspect, a portion of interest of the Acupuncture-MRI Probe needle antenna 101 material can be converted to a ceramic or another non-metallic tip 102, e.g., by oxidizing the desired portion of a metal Acupuncture-MRI Probe needle antenna 101. In certain aspects, a ceramic tip 102 on the Acupuncture-MRI Probe needle antenna 101 can be a capacitor dielectric that can also be designed and used as a capacitive tuning element.

As can be seen in FIG. 1, the Acupuncture-MRI Probe needle antenna 101 is essentially a linearly extending member having a pointed (ceramic) tip 102 on one end. Further in this aspect depicted in FIG. 1, the needle antenna 101 is coupled to tuning and matching non-magnetic ceramic capacitors 103 and 104, respectively positioned on the same or opposing sides of the needle antenna 101, which can connect the radiofrequency signal cable 107 to the needle antenna 101. It is understood that tuning and matching non-magnetic ceramic capacitors 103 and 104, respectively are used to adjust the resonance frequency and impedance parameters of the Acupuncture-MRI Probe 100 to desired values. The tuning and matching non-magnetic ceramic capacitors 103 and 104, respectively can be miniature ceramic non-magnetic or other suitable types with capacitance values that range from about 0.001 pF to 10 nF or from about 0.01 pF to 1 nF or from about 0.1 pF to 0.1 nF. The tuning non-magnetic ceramic capacitor 103 is commercially available as known to one skilled in the art. Further, a radiofrequency signal cable 107 is coupled to the tuning non-magnetic ceramic capacitor 103 and the needle antenna 101. As discussed below, in certain aspects, the Acupuncture-MRI Probe 100 may not necessarily require physical coupling to the radiofrequency signal cable 107 and/or may not require separate non-magnetic ceramic capacitance elements 103 and 104.

In certain aspects, the signal cable can be a radiofrequency signal cable 107 connected to a radiofrequency imaging console, such as an MRI or NMR console. Such radiofrequency signal cables and consoles are commercially available and known to one skilled it the art.

As can also be seen in the Acupuncture-MRI Probe 100 depicted in FIG. 1, the needle antenna 101 may be coupled to a mechanical actuator 109, which can control the longitudinal positioning of the needle antenna 101, e.g., to probe into the dermis. The mechanical actuator 109 can be coupled to the needle antenna 101 in any manner known to one skilled in the art. In certain aspects, such as that depicted in FIG. 1, the needle antenna 101 and non-magnetic ceramic tune and match capacitors 103 and 104, respectively can be coupled to an electromechanical piston 111 located in a hollow tube guide 110 that is, in turn, coupled to the mechanical actuator 109. The mechanical actuator 109 can be any commercially available mechanical actuator system, e.g., a coil and magnet system.

In operation, the Acupuncture-MRI Probe 100 can generate and detect localized radiofrequency magnetic fields along a radial coordinate. As depicted in FIG. 1, once the mechanical actuator 109 has longitudinally positioned at least a portion of the Acupuncture-MRI Probe needle antenna 101 in or adjacent the suspect tumor cells at a first position, the Acupuncture-MRI Probe 100 can detect radiofrequency field strengths as imaginary concentric cylindrical shells that are representations of rotating-frame imaging (RFI) voxels of variable radiofrequency field strengths. Further, each of these radial positions from the long axis of the Acupuncture-MRI Probe needle antenna 101 exhibits its magnetic resonance spectra. Thus, at a single radial position, the Acupuncture-MRI Probe 100 can provide data on the extent or size of the tumor radially extending out from the long axis of the Acupuncture-MRI Probe needle antenna 101. For example, the outer voxel may depict spectra (e.g., chemical shifts) containing a significant healthy tissue peak compared to a minimal if any tumor tissue peak. A mid-shell voxel may depict spectra where there is a greater tumor tissue peak than a healthy tissue peak at this voxel. Further, an inner-shell voxel may depict a larger tumor tissue peak than the healthy tissue peak at this voxel (and a larger tumor tissue peak at the inner-shell voxel compared to the mid-shell and outer-shell voxels).

In certain aspects, the spectra obtained from these measurements can include hydrogen and/or phosphorous chemical shifts, which may allow for the differentiation of tumor tissue from healthy tissue. It is appreciated by one skilled in the art awareness of chemical shifts indicative of healthy tissue and chemical shifts indicative of tumor tissue. See, for example, Taylor D G, Bore C F, “A review of the magnetic resonance response of biological tissue and its applicability to the diagnosis of cancer by NMR radiology,” J Comput Tomogr. 1981 June; 5(2):122-33; and Janna P. Wehrle, Cynthia Paella Martin, Jerry D. Glickson “NMR Spectroscopy and Its Application to the Study of Cancer,” a chapter of a book entitled: Innovations in Diagnostic Radiology, Part of the series Medical Radiology pp 93-116, ISBN no. 978-3-642-83415-8.

Further, in operation, the mechanical actuator 109 can then reposition the needle antenna 101 to a second position, e.g. deeper into the dermis, where additional spectra can be obtained at each of the imaging voxels, which can be repeated as many times as necessary to measure a thickness of the tumor or its level of penetration into the dermis. In such aspects, the mechanical actuator 109 can move the needle longitudinally over a range of about 1 mm to about 10 mm, or about 1 to about 5 mm.

In aspects, the radiofrequency field is transmitted to the Acupuncture-MRI Probe needle antenna 101 in discontinuous bursts or pulses. In such aspects, the pulse duration can be an amount of time ranging from about 0.1 microseconds (μs) to about 1500 milliseconds (ms), or from about 1 μs to about 500 μs, or from about 10 μs to about 100 μs. In certain aspects, the radiofrequency field is transmitted to the Acupuncture-MRI Probe needle antenna 101 having a certain pulse duration such that the diagnostic imaging procedure can be done without damaging the tissue of the subject, such as using the pulse durations mentioned immediately above.

In certain aspects, the Acupuncture-MRI Probe 100 can be utilized to treat cancerous cells, e.g., by supplying a longer pulse duration than that utilized for imaging, as discussed above. In such aspects, these longer pulse durations may affect cell necrosis to the cancer cells adjacent or near the needle antenna 101, with the energy of the pulses diminishing further away from the needle antenna 101. In such aspects, the pulse duration for treatment can range from about 0.1 microseconds (μs) to about 30,000 milliseconds (ms), or from about 100 ms to about 5000 ms. In certain aspects, the pulse durations for treatment may manifest into an energy volume of about 0.1 microjoules per mm³ (μJ/mm³) to about 1000 Joules per mm³ (J/mm³). In certain aspects, shorter duration pulses may affect cellular apoptosis. In one aspect, the shorter duration pulses can be shorter than one or more of the ranges discussed above for effective cellular necrosis.

In one or more aspects, during imaging operations one or more Acupuncture-MRI Probe needle antennae 101 may be simultaneously utilized. For example, in one aspect, a plurality of Acupuncture-MRI Probe needle antennae 101 may be coupled to a patch that can be placed on a subject effecting easy and quick placement on the subject. In such aspects, the Acupuncture-MRI Probe needle antennae 101 can be relatively short in lengths, such as about 1 to about 10 mm, or about 1 to about 5 mm.

As discussed above, in certain aspects, the Acupuncture-MRI Probe needle antenna 101 is physically coupled to radiofrequency signal cable 107 and one or more non-magnetic ceramic capacitors 103 and 104. In one alternative aspect, the Acupuncture-MRI Probe needle antenna 101 can be designed to be a self-resonator that can couple to an MRI radiofrequency coil through space and thereby not require a radiofrequency signal cable connection and can be a stand-alone resonating structure (in such aspects, the Acupuncture-MRI Probe needle antenna 101 may exhibit inherent capacitance through its structure, or alternatively the Acupuncture-MRI Probe needle antenna 101 may be physically connected to one or more non-magnetic ceramic capacitors 103 and 104).

What is claimed is:

1. An MRI detector comprising:
an Acupuncture-MRI Probe comprising a conductive material and one or more non-magnetic capacitance elements, the Acupuncture-MRI Probe needle antenna comprising a first portion having a thickness of from about 5 μm to about 1000 μm, wherein the Acupuncture-MRI Probe needle antenna exhibits an inductance of about 10⁻⁶ to about 10⁻¹² henry (H), wherein the Acupuncture-MRI Probe is configured to resonate when exposed to radiofrequency pulses from an MRI or NMR device thereby generating one or more localized radiofrequency magnetic fields with gradient distribution surrounding at least a portion of the Acupuncture-MRI Probe needle antenna.

2. The MRI detector according to claim 1, wherein the one or more non-magnetic capacitance elements are physically coupled to the Acupuncture-MRI Probe needle antenna.

3. The MRI detector according to claim 1, wherein the one or more non-magnetic capacitance elements form a second portion of the Acupuncture-MRI Probe needle antenna and are integral with the conductive material.

4. The MRI detector according to claim 3, wherein the Acupuncture-MRI Probe needle antenna is a flexible electrically non-conducting material that is coated with a conductive material.

5. The MRI detector according to claim 4, wherein the Acupuncture-MRI Probe needle antenna is coated with a 1-50 μm-thick layer of gold.

6. The MRI detector according to claim 1, wherein the Acupuncture-MRI Probe needle antenna is a hollow electrically-conductive tube with open top and bottom ends.

7. The MRI detector according to claim 6, wherein the Acupuncture-MRI Probe needle antenna is fabricated from one or more of the following materials: phosphor bronze, gold, platinum, non-magnetic stainless steel, copper, aluminum, silver.

8. The MRI detector according to claim 1, wherein the Acupuncture-MRI Probe needle antenna is a hollow electrically-conductive tube with perforated walls.

9. The MRI detector according to claim 8, wherein the Acupuncture-MRI Probe needle antenna is fabricated from one or more of the following materials: phosphor bronze, gold, platinum, non-magnetic stainless steel, copper, aluminum, silver.

10. A method for detecting cancer cells comprising:
contacting a subject with an Acupuncture-MRI Probe, the Acupuncture-MRI Probe comprising a conductive material and one or more non-magnetic capacitance elements, wherein the Acupuncture-MRI Probe needle antenna exhibits an inductance of about 10⁻⁶ to about 10⁻¹² henry (H); exposing the Acupuncture-MRI Probe needle antenna to one or more radiofrequency pulses so that the Acupuncture-MRI Probe needle antenna can generate one or more localized radiofrequency magnetic fields with gradient distribution surrounding at least a portion of the Acupuncture-MRI Probe needle antenna; detecting at least a portion of the localized radiofrequency magnetic fields along a radial coordinate; and determining one or more chemical shifts and associated quantities based on the portion of the localized radiofrequency magnetic fields.

11. The method according to claim 10, wherein the contacting comprising inserting at least a portion of the Acupuncture-MRI Probe needle antenna into the subject's skin.

12. The method according to claim 10, wherein each of the one or more radiofrequency pulses have a duration of from about 0.1 microseconds (μs) to about 1500 milliseconds (ms).

13. The method according to claim 10, wherein the procedure for obtaining images also incorporates pulsed magnetic field gradients generated by an MRI scanner that modify the static magnetic field to encode nuclear spins with spatial information.

14. The method according to claim 10, wherein the procedure for obtaining images incorporates advanced RFI methods.

15. The method according to claim 10, wherein the procedure for obtaining images incorporates advanced RFI methods and pulsed magnetic field gradients generated by an MRI scanner that modify the static magnetic field to encode nuclear spins with spatial information.

16. A method for treating cancer cells comprising:
contacting a subject with an Acupuncture-MRI Probe, the Acupuncture-MRI Probe comprising a hollow conductive material and one or more non-magnetic capacitance elements, wherein the Acupuncture-MRI Probe hollow needle antenna exhibits an inductance of about 10⁻⁶ to about 10⁻¹² henry (H); exposing the Acupuncture-MRI Probe hollow needle antenna to one or more radiofrequency pulses so that the Acupuncture-MRI Probe hollow needle antenna can generate one or more localized radiofrequency magnetic and electric fields with gradient distribution surrounding at least a portion of the Acupuncture-MRI Probe hollow needle antenna; detecting at least a portion of the localized radiofrequency magnetic fields along a radial coordinate; determining one or more chemical shifts and associated quantities based on the portion of the localized radiofrequency magnetic fields; applying radiofrequency pulses to the Acupuncture-MRI Probe hollow needle antenna to generate one or more localized radiofrequency electric fields to cause local heating and destruction of skin tumors; injecting imaging agents to enhance contrast of RFI and MRI images; injecting treatment agents and compounds into tumors to cause the destruction of tumors; and aspirating skin tumor tissue and associated materials to cause the removal of skin tumors.

17. The method according to claim 16, wherein the methods for RFI/MRI imaging and treatment/extraction of tumors occurs sequentially or simultaneously.

18. The method according to claim 16, wherein the methods for treatment/extraction of tumors includes the injection into tumors of cancer-destroying agents and compounds known in the art.

19. The method according to claim 18, wherein the cancer-destroying agents and compounds known in the art include: nanoparticles, porous wall hollow glass microspheres, radioactive materials, organometallic compounds, organic solvents, inorganic acids, inorganic bases, heat-generating compounds, salinomycin, cisplatin, nonplatinum-based halogenated molecules, reactive agents.

20. The method according to claim 19, wherein the porous wall hollow glass microspheres contain one or more of the following cancer destroying agents: radioactive elements, cancer drugs, salinomycin, organic solvents, inorganic acids, inorganic bases, heat-generating compounds, cisplatin, nonplatinum-based halogenated molecules, reactive agents.
